(19) United States
(12) Patent Application Publication
(10) Pub. No.: US 2003/0220650 A1
(43) Pub. Date: Nov. 27, 2003

(54) Minimally invasive bone manipulation device and method of use

(76) Inventors: Eric D. Major, Ashburn, VA (US); Richard W. Woods, Catonsville, MD (US); Mohit Bhatnagar, Gaithersburg, MD (US); Scott A. Jones, McMurray, PA (US)

Correspondence Address: Supervisor, Patent Prosecution Services, PIPER RUDNICK LLP, 1200 Nineteenth Street, N.W., Washington, DC, 20036-2412, US

(21) Appl. No.: 10/389,818

(22) Filed: Mar. 18, 2003

Related U.S. Application Data
(60) Provisional application No. 60/365,026, filed on Mar. 18, 2002.

(51) Int. Cl. A61B 17/58

(52) U.S. Cl. 606/90

(57) ABSTRACT

A novel surgical apparatus for use in orthopedic surgery procedures and a method for use is provided. The present invention relates primarily to the treatment of traumatic, pathogenic, or osteoporotic bone conditions of human and other animal body systems and, more particularly, to a novel apparatus and method for manipulating the vertebral body through a less invasive, percutaneous, surgical approach.

This application claims priority from U.S. Provisional Application Serial No. 60/365,026 filed Mar. 18, 2002. The entirety of that provisional application is incorporated herein by reference.

### BACKGROUND OF THE INVENTION

1. Field of the Invention

This invention relates to a novel surgical apparatus for use in orthopedic surgery procedures. Particularly, the present invention relates to the treatment of traumatic, pathogenic, or osteoporotic bone conditions of human and other animal body systems and, more particularly, to a novel apparatus and method for manipulating the vertebral body through a less invasive, percutaneous, surgical approach.

2. Background of the Technology

Surgical procedures frequently require that after surgical entry has been made the tissues within the patient's body must be expanded away from the surgical point of interest in order to provide better access and visibility for the surgeon. Various surgical devices have been developed to assist the surgeon to displace tissue and expand the interior cavities of the body during both open surgical procedures and less invasive surgical procedures.

Surgical procedures vary dramatically depending upon the region of the body involved and the purpose of the surgery. The design and the use of surgical instruments are equally specialized and instruments which seemingly are intended for similar purposes can be significantly different from one another depending upon the exact nature of the surgery. The use of retractors to expand an opening or create a space to facilitate access for the surgeon during the performance of a surgical procedure is well known in the art. It is, however, often the case that one tissue retractor designed for use in a particular surgical procedure will be completely unsuitable for retraction of tissue in a different type of surgery.

Conventional surgical procedures, which have been employed to alleviate vertebral compression failures, involve major invasive surgical techniques with all of the possible negative consequences. The close proximity of the spinal cord to damaged sections of a vertebra, which are in need of surgical repair, presents a particularly hazardous situation for the patient and increases the challenge for the surgeon tremendously. Recent surgical innovations have been made that provide a means of reinforcing damaged vertebra by the injection of bone cement into the vertebral body. However, in patients suffering from crippling effects of osteoporosis and the possibility of compression fractures of multiple vertebra, there remains a need for an effective and safe way to distract the vertebral endplates so as to alleviate the compressed disposition of the vertebral body. Such a specialized requirement dictates the need for a specialized tissue expansion device. Numerous tissue expansion devices have been developed for different surgical procedures but at present none are suitable or capable of adaptation so as to safely manipulate the endplates of a patient's vertebra.

U.S. Pat. No. 6,309,349 issued to Bertolero et al. discloses a typical tissue retractor used to expand a body wall entry incision. For nearly a century such retractors with one or more specialized modifications have been used to meet the particular requirements for body wall incision expansion in different surgical procedures. Examples of other retractor-type tissue manipulators or expanders are disclosed in U.S. Pat. No. 6,354,994 issued to Rullo et al. and U.S. Pat. No. 6,322,500 issued to Sikora et al. Retractors representative of this type are commonly used for open surgical procedures and would be unsuited to less invasive surgical procedures such as, for example, endoscopic surgery.

U.S. Pat. No. 6,319,252 issued to McDevitt et al. discloses an apparatus designed to attach a patient's soft tissue to bone material. The design of the apparatus permits the user to manipulate an expander pin through soft tissue and into a position in the bone to which the tissue is to be anchored. A screw-type expander mechanism allows the diameter of the circumferentially disposed expander pin to be increased so as to press into the surrounding bone. A small portion of bone is equilaterally displaced around the circumference of the expander pin. A very similar device designed for a slightly different surgical procedure is disclosed in U.S. Pat. No. 6,221,107 issued to Steiner et al. The device of Steiner et al. also employs a screw-type expander provided with special features, which facilitate the attachment of the ligament of a patient to a passage or channel created in a bone by the surgeon. Such devices have vary specialized uses and are incapable of selectively directing the expansion of the surrounding tissue. Further with a screw-type expander the amount of expansion of the surrounding tissue is very limited; as such this type of expansion device is typically suited only for anchoring the device into the surrounding tissue.

U.S. Pat. No. 5,888,196 issued to Bonutti discloses a mechanically expandable retractor for use in arthroscopic surgery. This surgical device is a dual sleeve-type expander, which is designed for creating a void in subsurface tissues using arthroscopic surgical procedures. This type of device is designed for movement of tissue in the repair of carpal tunnel syndrome. The dual sleeve expansion device of Bonutti can be configured to have one or more expandable portions of the flexible outer sleeve located near the tip of the device. The jointed expandable portions present an acute outermost contact surface that could easily damage tissue with which it comes in contact and further would provide a limited point of expansion rather than a uniform area of expansion. While the expanding sleeve of Bonutti provides an expansion device that can be used in less invasive surgical procedures such as treatments for carpal tunnel syndrome, it would be wholly unsuited for safe use in the manipulation of bone, particularly when employed within a vertebral body, where the point-oriented expansion device might seriously damage already weakened bone. U.S. Pat. No. 6,139,508 issued to Simpson et al. discloses a similar sleeve expansion device for displacing tissue in the vicinity of a biopsy site. The sleeve expansion design devices have limited and specialized use for the temporary dislocation of soft tissue in the vicinity of a surgical site or a biopsy site. WO 02/13700 A2 discloses a flexible sleeve expansion device having a deformable flexible distal portion of the sleeve for use in treatment of the spine. This device, much like balloon technology relies upon the radial displacement of a soft flexible sleeve, however, it does not have the benefit of the compressed air or fluid that provides a more consistent outward force in balloon type devices.

U.S. Pat. No. 6,358,266 issued to Bonutti discloses an active cannula or sleeve, which can be used to enlarge a channel so as to enable the positioning of a scope or instrument or to move or relocate tissue. Bonutti '266 employs an inflatable balloon as the mechanical device to expand the skin around a surgical entry site. The application of balloon technology as a tissue expander has also been employed in U.S. Pat. No. 6,241,734 issued to Scribner et al., which teaches the use of a balloon expansion device employed with a vertebra for the purpose of creating a space with the tissue. The Scribner et al. device, like other balloon-type expansion devices are uncontrolled and multi-directional, which can lead to undesirable expansion of the bone or bone fragments being displaced in unwanted directions.

Conventionally used tissue expansion devices are each configured to precisely meet the specific needs for particular surgical procedures and, as such, are not readily (if at all) adaptable to meet the needs of a different surgical procedure.

There is therefore a need for a less invasive surgical device, which can be precisely controlled by the surgeon to expand tissue and transmit sufficient force to manipulate bone in a selected direction or, if desired, in multiple directions. None of the tissue expansion devices currently used to assist a surgeon in creating a void in tissue can fulfill this requirement.

### SUMMARY OF THE INVENTION

The present invention provides a device and method, which can be used by a surgeon to meet the above identified need for specialized surgical procedures. In particular, the device of the present invention is designed for use as a less invasive means of controllably manipulating a damaged bone, and in particular a patient's vertebra to create a void therein so as to enable a surgeon to accomplish a surgical procedure.

It is therefore an object of the present invention to provide a tissue expansion device, which can be used in open or minimally invasive surgical procedures to selectively, in a measured manner, directionally expand tissue to create a void with a tissue mass, particularly within the interior of the bone.

It is a further object of the present invention to provide a tissue expansion device that is capable of endoscopic or arthroscopic use by a surgeon to controllably create with specificity a void within the vertebral body of a subject's vertebra.

It is another object of the present invention to provide a tissue expansion device that is capable of selectively exerting sufficient force in one or more directions to manipulate a tissue mass, particularly bone tissue.

Another object of the present invention is to provide a tissue expansion device that is capable of the controlled, measured and predictable distracting of the vertebral endplates of a patient's vertebra from within the vertebral body.

Another object of the present invention is to provide a tissue expansion device, which incorporates a selectively releaseable spreading assembly or modular expansion member for correcting the positioning of tissue or bone. After the tissue or bone correction has been effected the spreading assembly can be released and left implanted in the patient to maintain the correction.

Another object of the present invention is to provide a method for forming a space within tissue while employing a minimally invasive surgical procedure.

Another object of the present invention is to provide a method for manipulating bone tissue in a directionally controlled and measured manner.

Another object of the present invention is to provide a method for distracting the vertebral endplates of a patient's vertebra from with the vertebral body.

Another object of the present invention is to provide a method for correcting the positioning of tissue or bone with a patient and implanting a spreading assembly, modular expansion member, into the patient to maintain the tissue position correction.

Another object of the present invention is to provide a method for providing additional long term support for the vertebral body by inserting supporting material, such as bone cement or the like, into the space formed by the device of the present invention. This object can be achieved after the device of the present invention has been used to create a space within the veterbral body and the spreading assembly has been removed. Alternatively, this object can be achieved by inserting the bone cement into the space within the vertebral body after the spreading assembly has been selectively detached from the shaft assembly of the device. In this embodiment, the spreading assembly is left in place within the vertebral body so as to provide additional support along with the bone cement for the vertebra.

All of these objects are achieved by the use of the device and method of the present invention.

### BRIEF DESCRIPTION OF THE DRAWINGS

The invention will now be described, by way of illustration only, with reference to the accompanying drawings.

FIG. 1 is a cross-sectional plan view of a first embodiment of the device of the present invention, which shows the mechanism configured in an expanded position.

FIG. 2 is a cross-sectional plan view of a first embodiment of the device of the present invention, which shows the mechanism configured in an closed position.

FIG. 3 is an isometric view of the mechanical detail of the modular expansion member of a second embodiment of the present invention in the expanded position.

FIG. 4 is an isometric view of the mechanical detail of the modular expansion member of the embodiment of the present invention of FIG. 3 shown in the closed position.

FIGS. 5A-C show plan, top and end views of the modular expansion member of the embodiment of the present invention shown in FIGS. 3-4.

FIG. 6 is an isometric view of an alternative handle member of the present invention.

FIGS. 7A-B show a side and end view of a third embodiment of the device of the present invention in a closed (A) and open (B) position.

FIGS. 8A-B show a side and end view of a fourth embodiment of the device of the present invention in a closed (A) and open (B) position.

### DETAILED DESCRIPTION OF THE INVENTION

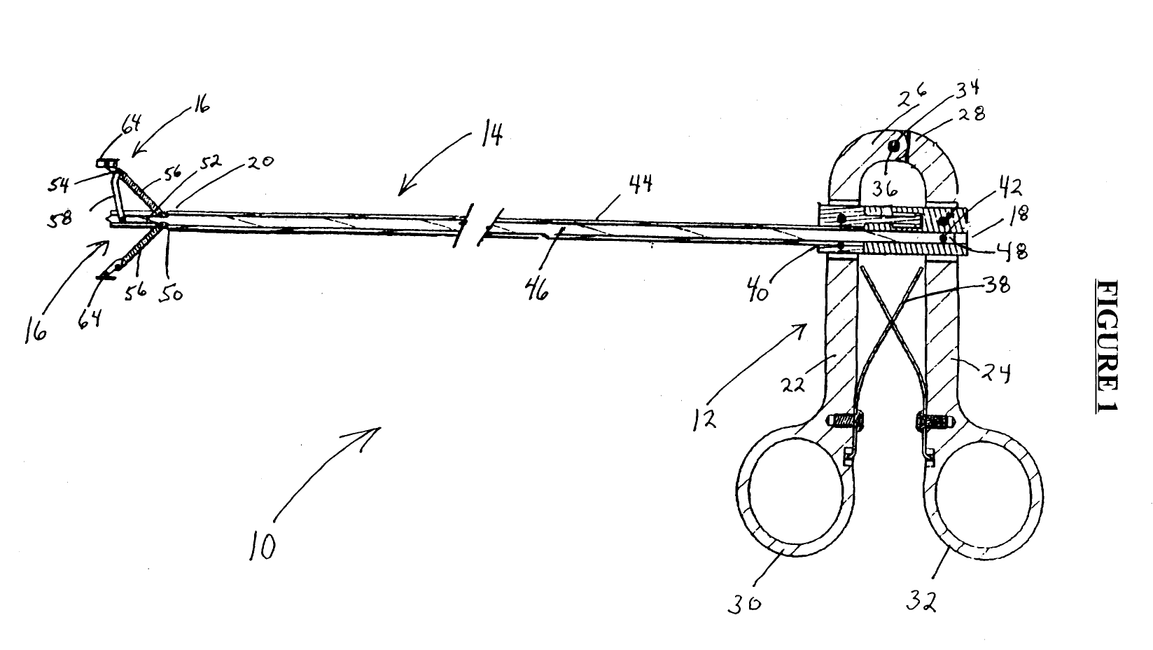

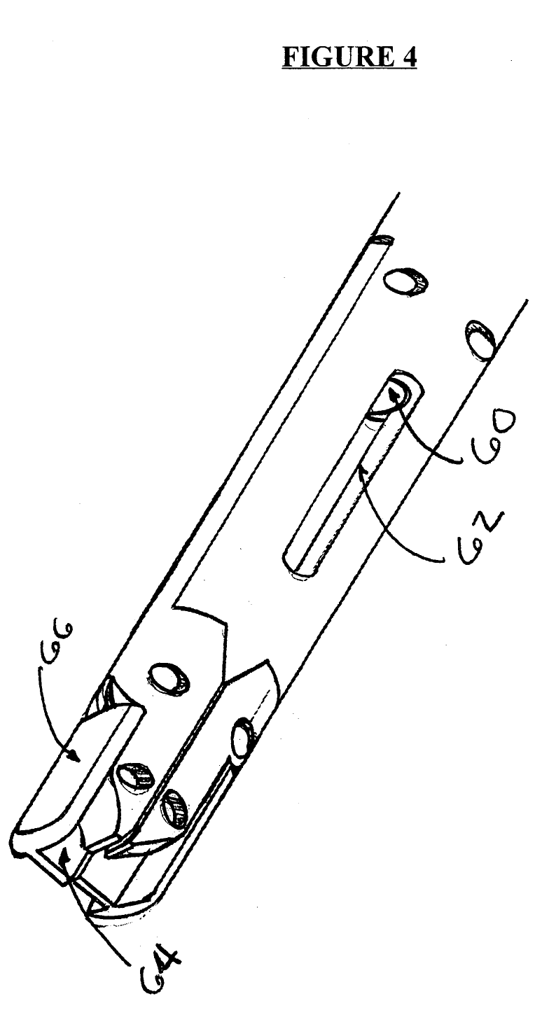

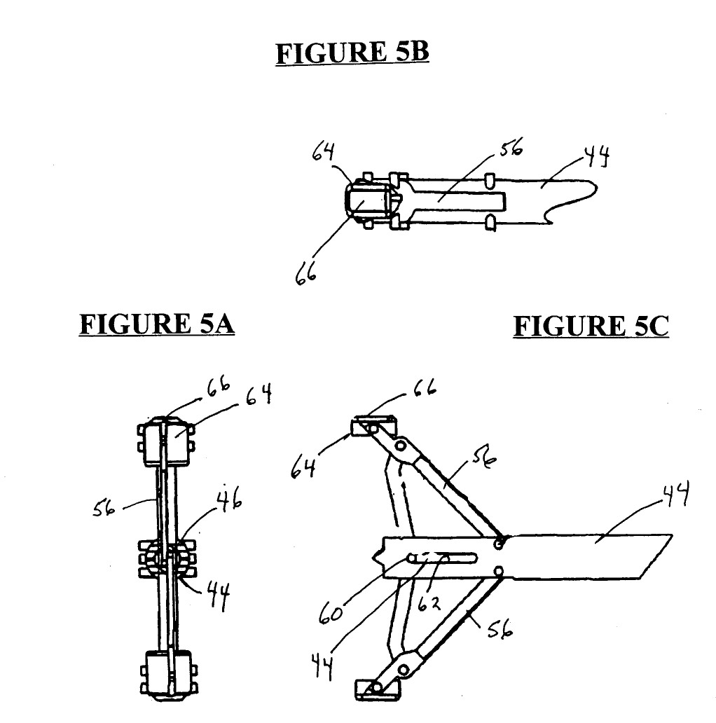

The apparatus and method of the present invention can be adapted for use in a variety of surgical procedures which require precise tissue manipulation by a surgeon. The following description and associated FIGS. 1-9 are provided as non-limiting examples of the invention, which is defined with particularity only by the claims of the present invention.

As shown in FIGS. 1-7, a tissue manipulation device (10) is provided. A preferred embodiment of the present invention is configured to be particularly well-suited to the manipulation of bone in a subject. By way of example, the device (10), as best shown in FIGS. 1-5, includes three portions or assemblies which can be integrally formed or separately fabricated prior to being joined to form the device (10).

The three assemblies, which together form the device (10), include a handle assembly (12), a shaft assembly (14) and a spreading assembly (16). A proximal end (18) of the shaft assembly (14) is connected to the handle assembly (12) and a distal end (20) of the shaft assembly (14) is connected to the spreader assembly (16).

As earlier indicated, these three assemblies (12, 14, 16) can be integrally formed or, alternatively, can be individually formed and then connected to form the whole device (10). It is also within the concept of the present invention for the one or more of the assemblies to be releasably connected one to the other.

In an embodiment where the three assemblies (12, 14, 16) are formed and subsequently permanently connected one to the other, the connections may be of any character known for connecting parts of a whole into one unit, to include, for example: gluing, soldering, welding, mechanically joining by rivets or screws or the like or any other means for permanently connecting parts one to the other.

If the three assemblies (12, 14, 16) are releasably attached one to the other, any releasable attachment means known can be used, to include, for example: snap fittings, bayonet fittings, luer lock fittings, threaded fittings, cotter-pin connections, plug and socket connections, or any other releasable attachment means. If the device (10) is integrally formed, any manufacturing process known can be employed, to include extrusion molding, die-casting, tooling, or any other means of fabricating such a device.

When the device (10) is configured to permit releasable attachment of the three assemblies (12, 14, 16) to each other, it is possible for the user to attach alternative embodiments of any of the three individual assemblies (12, 14, 16). This feature of the invention permits each of the three assemblies (12, 14, 16) to separately have specifically designed embodiments that are configured for very specific uses or for individual preferences of the using surgeon. Thus, one embodiment of the device (10) enables the user to customize the combination of the three assemblies (12, 14,16) to meet the particular requirements for a specific surgical procedure.

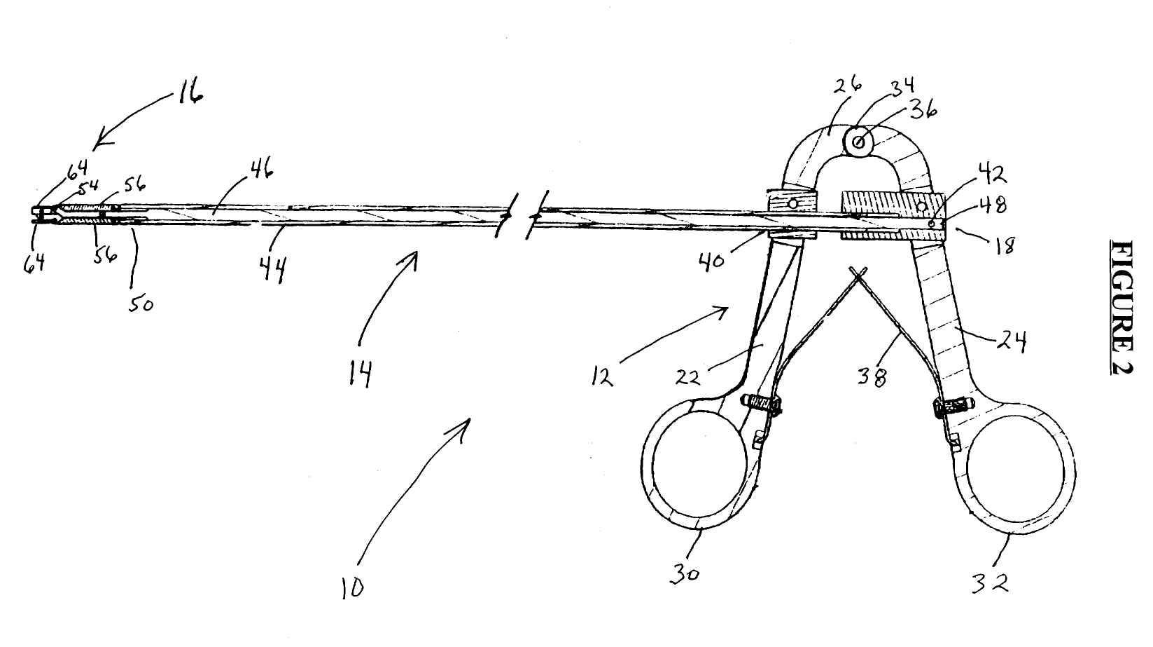

As best shown in FIGS. 1-2, the handle assembly (12) can be configured to operate as a scissor-like embodiment so as to provide ease of handling by a user during operation of the device (10). FIG. 1 provides a depiction of the scissor-like embodiment of the handle assembly (12) in the device-open configuration while FIG. 2 depicts this embodiment in a device-closed configuration. It is within the concept of the invention to reverse the operable effect of the handle positions shown in FIGS. 1 and 2 without departing from the concept of the invention. The scissor-like embodiment of the handle assembly (12) depicted in FIGS. 1 and 2 can include a first handle member (22) and a second handle member (24), each handle member (22, 24) having respectively a connecting end (26, 28) and a gripping end (30, 32).

Each of the connecting ends (26, 28) are movably connected one to the other by a handle member connector (34). In a preferred embodiment, the handle member connector (34) is configured as a handle member pivot pin (36) although the handle member connector (34) can be configured as rotationally engaged portions of the first and second handle members (22, 24). Thus, the handle member connector (34) can be configured to include a pivot pin (36), a rotating ball-and-socket joint, a rotating rim-and-groove joint, or any other connection design which permits the connecting ends (26, 28) of the first and second handle members (22, 24) to be connected in a moveable relationship one to the other. The first and second handle members (22, 24) can be biased into a closed or open relationship one to the other by a biasing member (38), which can be releasably attached to facilitate repair and replacement.

The handle members (22,24) can define a actuating arm portal (40) which provides for an arm anchor (42) defined within the second handle member (24). The portion of the portal (40) defined through the first handle member (24) can be sized and configured to hold and restrict the movement of an actuating arm sleeve (44). The shaft assembly (14) includes the actuating arm sleeve (44) and the actuating arm (46). The actuating arm sleeve (44) can be sized and configured to permit sliding passage of the actuating arm (46). The actuating arm (46) at its proximal end (48) can be releasable attached to the actuating arm anchor (42). Both the actuating arm (46) and the actuating arm sleeve (44) are configured to be in a sliding relationship, one within the other, and to be of a respective fixed length such that the actuating arm (46) can slide to a position which extends beyond the distal end (50) of the sleeve (44).

Attached at the distal end of the actuating arm (52) is a spreading assembly (16). The spreading assembly (16) can be sized and configure to fit within the sleeve (44) when the actuating arm (46) is fully withdrawn into the sleeve (44). The spreading assembly (16) can be releasable from the actuating arm to permit the surgeon to insert the spreading assembly into a bone, actuate the spreading assembly into an expanded position, and, if desired, disconnect the spreading assembly so as to permit it to be left within the bone. This optional releasable feature of the spreading assembly (16) also permits the device to be configured with different sizes and configurations of spreading assembly prior to use.

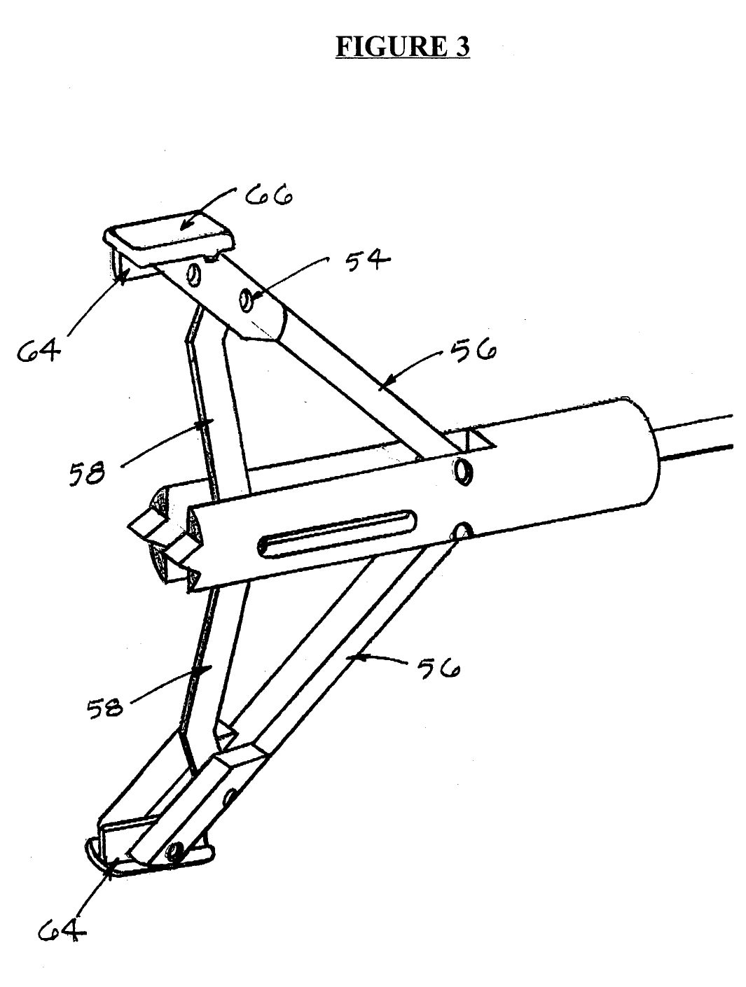

The spreading assembly (16), as best seen in FIGS. 3, 4, and 5A-C, can be configured to include at least one extending arm (56) that can be in pivotal relationship at one end with the actuating sleeve (44). Any sufficiently strong material compatible for use in surgical instruments can be used in the manufacture of the device. The extending arm (56) is preferably manufactured of a rigid material to provide strength and reliable, consistent performance during operation. Conventional spreaders, which can include flexible sleeves are incapable of providing the even, consistent force during operation that is essential for safely manipulating bone material. This especially important when manipulating the end plates of a vertebral body. At least one bracing member (58) is rotationally attached at one end to the actuating arm (46) and rotationally attached at the other end to the proximal end of the extending arm (56). Due to the operational requirement of manipulating bone, it is preferable that the bracing member be manufactured of rigid material, however any material suitable for use in surgical instruments can be used if sufficiently strong. In operation, when the actuating arm (46) is extended distally beyond the confines of the sleeve (44), the bracing member (58) serves to exert a force on the extending arm (56) so as to force the spreading assembly into an open position. A locking mechanism can be provided so as to releasably lock the spreading assembly into position. This locking mechanism can be configured as a notch, slot, or other like means to fix the spreading assembly into an open position. The locking mechanism can be selectively locked or unlocked.

To facilitate smooth operation of the spreading assembly (16), a guide bar (60) sized and configured to slidably move with the confines of a guide slot (62) can be provided. The guide slot (62) can define the forward most and reward most movement of the guide bar (60) and in so doing control the degree of extension of the spreading assembly in operation. The guide slot (62) can thus be sized to control the amount of extension possible for the spreading assembly (16). The distal most portion of the extending arm (56) can be provided with a base plate (64), which can be pivotally attached thereto. The base plate (64) is sized and configured to provide a contact surface (66) that in operation is brought into contact with the bone to be manipulated. This contact surface (66) can provide a protective element to distribute the pressure exerted by spreading assembly across a broader surface of the bone being manipulated.

To provide visual feedback to the surgeon, the device (10) can be manufactured such that at least a portion of the device is radiopaque. It is within the concept of the invention that only select portions of the device (10), such as, for example, the contact surface (66) or the base plate (64) are manufactured or treated to include radiopaque material. Any method known in the art to manufacture or treat the device (10) so as to have a radiopaque quality can be employed without departing from the general concept of the present invention.

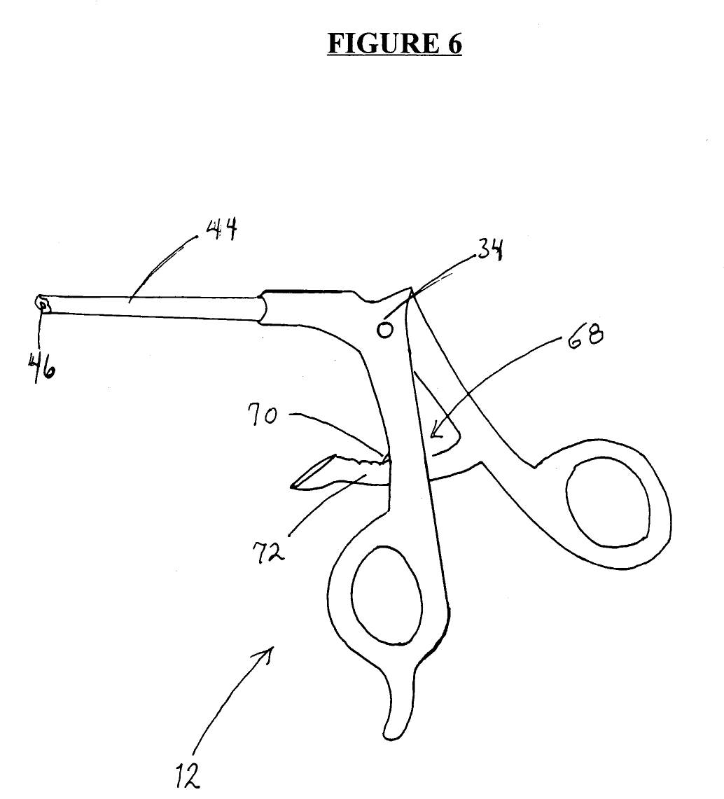

The first embodiment of the present invention described above illustrates the concept of the invention. It is, however, within the scope of the invention to configure the device (10) with a wide variety of handle assemblies (12) that would serve to actuate the device by movement of the actuating arm (46) with the actuating arm sleeve (44). Another non-limiting example of an alternative handle assembly (12) is shown in FIG. 6. This alternative handle assembly (12) can be configured similar to the scissor-like embodiment shown in FIGS. 1-2 with the additional feature of a handle locking assembly, generally shown at (68). The handle locking assembly (68) can be provided with a locking catch (70) similar to that typically found on a hemostat instrument. The locking catch (70) can be employed with a handle locking arm (72) that is configured for releasably locking the handle assembly (12) to a desired open, partially open, or closed position as desired by the using surgeon.

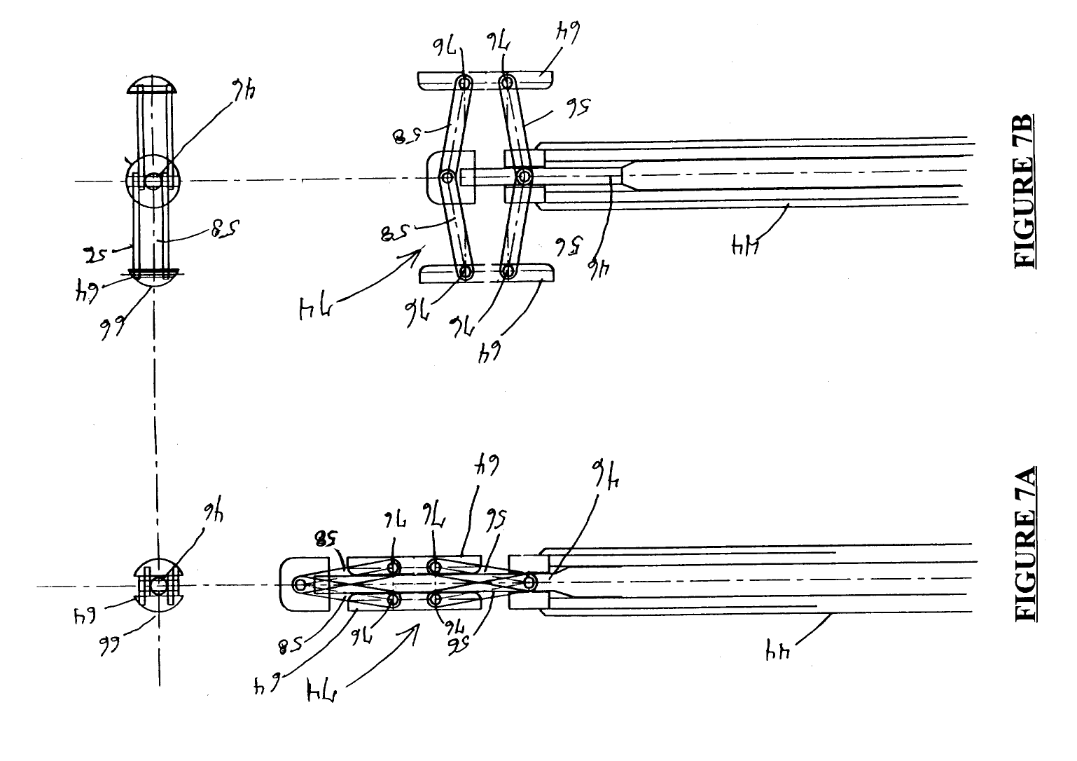

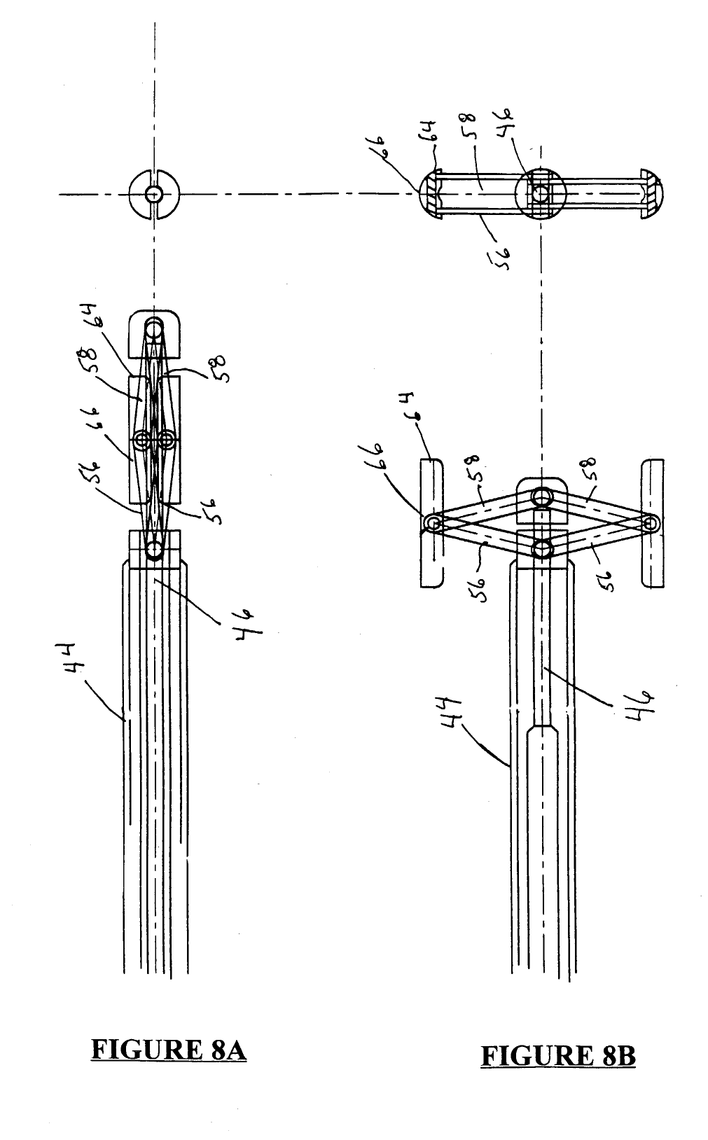

As best shown in FIGS. 7A-B and 8A-B, additional alternative embodiments of the present invention can be provided which employ the same concept of the earlier described embodiment with the modification of providing a dual-scissor assembly, generally shown at (74), that permits the use of two bracing members (58) pivotally attached to the actuating arm (46) at the proximal end and pivotally attached at a base plate pivot point (76). In FIGS. 7A-B, an alternative embodiment using two bracing members (58) attached at a single base plate pivot point is shown in both the closed (A) and open (B) positions. In FIGS. 8-A-B, the alternative embodiment is shown using two bracing members (58) attached a two distinct pivot points (76), one respectively for each bracing member (58). These alternative embodiments provide a configuration with the potential for increased leverage strength during the opening of the spreading assembly (16) as well as, in the case of FIGS. 7A-B, a broader base of support for the base plate (64).

Each of the embodiments described above are provided for illustrative purposes only and it is within the concept of the present invention to include modifications and varying configurations without departing from the scope of the invention that is limited only by the claims included herewith.

What is claimed is:

1. A surgical apparatus for use in manipulating tissue of a subject, comprising:
an elongated shaft assembly having a proximal end and a distal end, said elongated shaft comprising an actuating arm and an outer sleeve, said outer sleeve slidably circumscribing at least a portion of said actuating arm,
a handle assembly attached to said proximal end of said shaft assembly, said handle member being attached to said shaft assembly and configured such that actuation of said handle assembly is capable of effecting movement of actuating arm within said outer sleeve, and
a spreading assembly attached to said distal end of said shaft assembly, said spreading assembly comprising at least one bracing member and at least one extending arm, said bracing member having a first end and a second end, said first end being rotationally attached to said actuating arm and said second end being rotationally attached to said extending arm, such that upon movement of said actuating arm, said bracing member is capable of radially displacing at least a portion of said extending arm,
wherein said radially displaced extending arm when displaced is capable of contacting and manipulating said tissue.

2. The surgical apparatus of claim 1, wherein said tissue is within the vertebral body of said subject.

3. The surgical apparatus of claim 1, further comprising a base plate attached to said extending arm.

4. The surgical apparatus of claim 3, wherein said extending arm has a proximally disposed arm first end and a distally disposed arm second end, said base plate being attached at said arm second end.

5. The surgical apparatus of claim 4, wherein said base plate is rotationally attached to said extending arm.

6. The surgical apparatus of claim 1, wherein said spreading assembly is releasably attached to said shaft assembly.

7. The surgical apparatus of claim 5, wherein said spreading assembly can be locked in a position wherein said base plate is radially displaced from said shaft assembly.

8. The surgical apparatus of claim 1, wherein said bracing member is manufactured of rigid material.

9. The surgical apparatus of claim 1, wherein said handle assembly comprises a first handle member and a second handle member.

10. The surgical apparatus of claim 9, wherein said handle assembly further comprises a biasing member.

11. The surgical apparatus of claim 10, wherein said biasing member is capable of exerting a bias to keep said first handle member apart from said second handle member.

12. The surgical apparatus of claim 9, wherein said first handle member comprises a actuating arm anchor, said actuating arm being movably connected to said arm anchor.

13. The surgical apparatus of claim 12, wherein said second handle member comprises an actuating arm portal, said arm portal being connected to said outer sleeve; such that when said first handle member and said second handle member are moved in relation one to the other, said actuating arm slidably moved within said outer sleeve through said actuating arm portal.

14. The surgical apparatus of claim 1, wherein said spreading assembly comprises multiple extending arms and multiple bracing members.

15. The surgical apparatus of claim 14, wherein said extending arms are rotationally connected to a base plate.

16. The surgical apparatus of claim 15, wherein said base plate has two extending arms rotationally connected thereto at a single rotation point.

17. The surgical apparatus of claim 15, wherein said base plate has two extending arms rotationally connected at separate respective rotation points.

18. The apparatus of claim 1, wherein at least two of the following are releasably connected one to the other, said shaft assembly, said handle assembly and said spreading assembly.

19. The apparatus of claim 1, wherein said handle assembly further comprises a handle position lock.

20. The apparatus of claim 1, wherein at least a portion of said device is radiopaque.

21. A method of forming a space within a vertebral body of a subject, the method comprising:
providing an apparatus according to claim 1,
inserting said apparatus into the vertebral body of said subject,
actuating the handle assembly so as to cause the spreading assembly to open and contact the interior bone material of the vertebral body,
whereby said vertebral body has a space formed.

22. The method of claim 21, wherein said inserting step is accomplished using minimally invasive surgical technique.

23. The method of claim 21, where said spreader assembly of said apparatus according to claim 1 is disconnected and left within said vertebral body after said space is formed.

24. The method of claim 21, wherein said spreading assembly is removed from said vertebral body after said space is formed.

25. The method of claim 23, further comprising the step of filling said space formed with supporting material.

26. The method of claim 24, further comprising the step of filling said space formed with supporting material.
